(19) United States
(12) Patent Application Publication
(10) Pub. No.: US 2022/0175393 A1
(43) Pub. Date: Jun. 9, 2022

(54) TOURNIQUET DEVICE

(71) Applicant: NATIONAL CHENG KUNG UNIVERSITY, Tainan City (TW)

(72) Inventors: Wei-Fang Wang, Tainan City (TW); Yueh-Ju Liao, Tainan City (TW); Ying-Ju Chang, Tainan City (TW)

(21) Appl. No.: 17/528,235

(22) Filed: Nov. 17, 2021

(30) Foreign Application Priority Data
Dec. 3, 2020 (TW) 109142629

(51) Int. Cl. A61B 17/132; G16H 40/63

(52) U.S. Cl. CPC A61B 17/1327; A61B 2017/00119; G16H 40/63

(57) ABSTRACT

Provided is a tourniquet device including a belt body and a timer, wherein the timer includes: a housing having a plurality of bulges; a connecting component arranged on the outside of the housing to connect the belt body to the timer; and a timing unit and a warning unit, which are arranged inside the housing, wherein the belt body is clamped between the bulges and the connecting component. In the tourniquet device, the timer is small, simple to operate, and not easy to fall off from the belt body. In addition, the tourniquet device is suitable for users' conventional operation without undue difficulties in use.

### BACKGROUND

### 1. Technical Field

The present disclosure relates to a tourniquet device, and particularly to a tourniquet device having timing and warning functions.

### 2. Description of Relevant Art

Medical professionals often use tourniquets as auxiliary devices when performing blood drawing, intravenous injection, or hemodialysis to properly impose pressure on the puncture point of the patient to stanch bleeding, or properly congest blood vessels on the blood drawing site of the patient for inserting a needle. However, in the clinical practice, the tourniquet often fails to be properly removed from the patient after using due to medical professionals' complicated work or the patient's carelessness or unconsciousness, which causes the blood circulation of the patient to be blocked for too long, thereby resulting in medical injuries of traumatic compartment syndrome such as thrombus, swelling, paralysis, or tissue necrosis.

Presently, parts of commercial tourniquet devices are equipped with accessories such as alarms or timers. However, problems often occur in the clinical applications, such as accessories falling off from the tourniquet easily, accessories often being turned on inadvertently, oversized accessories resulting in abnormal sensation or discomfort sensation to the patients, complicated operation of accessories, or the users being forced to change original operation of the tourniquet in accordance with accessories. In addition, commercial tourniquet devices further exist circumstances such as insufficient warning effects or higher prices. Accordingly, there is an unmet need in the art to provide a tourniquet device that can solve the aforementioned problems.

### SUMMARY

In view of the above, the present disclosure provides a tourniquet device with a small size, economic benefits, simple operation, and high warning efficiency, which comprises a belt body and a timer, wherein the timer includes: a housing having a plurality of bulges; a connecting component arranged on outside of the housing to connect the belt body to the timer; and a timing unit and a warning unit arranged inside the housing, wherein the belt body is clamped between the bulges and the connecting component.

In at least one embodiment, the housing is provided with a recessed button to turn on or turn off the timing unit and/or the warning unit.

In at least one embodiment, the width of the timer is less than or equal to the width of the belt body.

In at least one embodiment, the connecting component has a first end and a second end opposite to the first end, wherein the first end is fixed with the housing, and a dimension of the bulges approaching the first end is greater than a dimension of the bulges approaching the second end.

In at least one embodiment, the dimension of the bulges is gradually increased in the direction from the second end extending to the first end.

In at least one embodiment, the timer is positioned at 1 cm to 5 cm from the terminal of the belt body.

In at least one embodiment, the housing includes water-proof material.

In at least one embodiment, the warning unit is at least one selected from the group consisting of a vibrator, a loudspeaker, and an illuminator.

In at least one embodiment, the sound made by the loudspeaker has a frequency below 20 kHz and/or a loudness below 90 dB, and the illuminator includes a red light-emitting diode (LED) and a green LED.

In at least one embodiment, the belt body is provided with a pressing block.

In at least one embodiment, the timer further comprises at least one selected from the group consisting of a wireless charging unit, a wireless communication unit, and a pressure sensing unit.

The present disclosure provides a timer that is small in size, simple to operate, and not easy to fall off from the belt body. Moreover, the tourniquet device of the present disclosure is suitable for original operation of the tourniquet user so as to reduce difficulties in use.

### BRIEF DESCRIPTION OF THE DRAWINGS

FIG. 1 illustrates a schematic view of the tourniquet device of an embodiment of the present disclosure.

FIG. 2 illustrates a top view of the tourniquet device of an embodiment of the present disclosure.

FIG. 3A illustrates a side view of the tourniquet device of an embodiment of the present disclosure.

FIG. 3B illustrates a side view of the tourniquet device of another embodiment of the present disclosure.

### DETAILED DESCRIPTION

The following descriptions of the embodiments illustrate implementations of the present disclosure, and those skilled in the art of the present disclosure can readily understand the advantages and effects of the present disclosure in accordance with the contents herein. However, the embodiments of the present disclosure are not intended to limit the scope of the present disclosure. The present disclosure could be practiced or applied by other alternative implementations, and every detail included in the present disclosure can be provided with alternative changes or modifications in accordance with different aspects and applications without departing from the scope of the present disclosure.

The features such as a ratio, structure, dimension shown in accompanying drawings herein are just used to accord with the contents disclosed in the present disclosure for those skilled in the art of the present disclosure to read and understand the present disclosure, rather than to limit the implemented scope of the present disclosure. Thus, under the circumstances without affecting the achievements and produced effects of the present disclosure, any changes of proportion relationship, structure modification, or dimension adjustment should fall within the scope of the technical contents disclosed in the present disclosure.

The phrase “comprise/comprising,” “include/including,” or “have/having” specific components described herein, unless otherwise explained, may include other components such as elements, ingredients, structures, regions, portions, devices, systems, steps, or connection relationships, etc., rather than excluding those other elements.

The phrases “on,” “under,” “front,” and “back” described herein are just used to clarify the embodiments of the present disclosure, rather than used to limit the implemented scope of the present disclosure, and under the circumstances without substantially changing the technical contents of the present disclosure, adjustments, exchanges, and modifications of relative positions and relationships thereof should be considered as the implemented scope of the present disclosure.

The sequential phrase “first” or “second” used herein are just used to describe or distinguish components such as elements, ingredients, structures, regions, portions, devices, systems, etc., rather than used to limit the implemented scope of the present disclosure or to limit the spatial order of those components. In addition, unless otherwise explanation, the singular forms “a” and “the” used herein include plural forms, and the phrases “or” and “and/or” used herein are interchangeable.

The numeral ranges used herein are inclusive and combinable, and any numeral value that falls within the numeral scope herein could be taken as a maximum or minimum value to derive the sub-ranges therefrom. For example, it should be understood that the numeral range “from 1 cm to 5 cm” comprises any sub-ranges between the minimum value of 1 cm to the maximum value of 5 cm, such as the sub-ranges from 1 cm to 4 cm, from 2 cm to 5 cm, and from 2.04 cm to 3.04 cm.

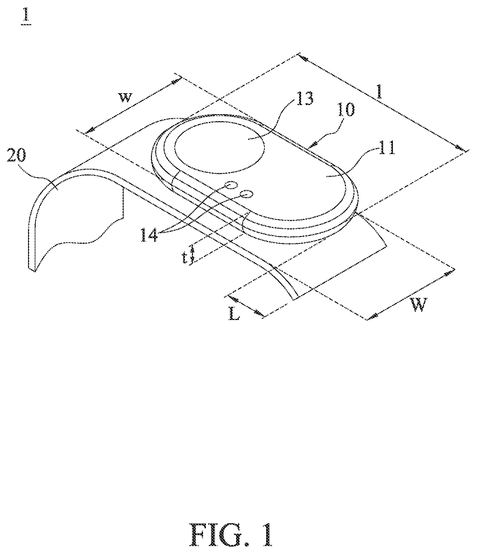

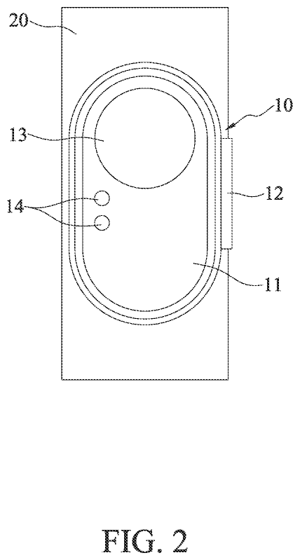

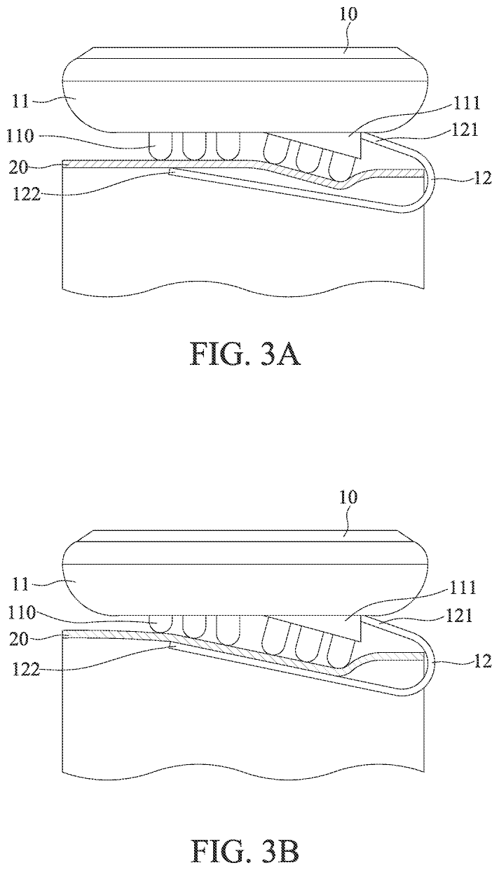

In at least one embodiment of the present disclosure, the tourniquet device 1 of FIG. 1, FIG. 2, and FIG. 3A comprises a timer 10 and a belt body 20, wherein the timer 10 comprises a housing 11 and a connecting component 12 arranged on outside of the housing 11, and the timer 10 connects to the belt body 20 by the connecting component 12.

The phrase “connect/connection” used herein refers to a plurality of elements directly or indirectly join together. The phrase “direct connection” refers to direct contact between a plurality of elements to join together, and the phrase “indirect connection” means that a plurality of elements are joined together by at least one connecting component. The means to achieve “connection” used herein comprises, but is not limited to, suturing, joining, bonding, embedding, screwing, fastening, nailing, clamping, attaching, puncturing, clipping, disposing, molding in one piece, or a combination of two or more thereof compactly or with a gap. The phrase “connecting component” used herein refers to the element that can achieve the aforementioned means to achieve “connection.”

For example, as shown in FIG. 3A, the connecting component 12 has a first end 121 and a second end 122 opposite to the first end 121, wherein the first end 121 is fixed with the back of the housing 11 to form an enclosed structure, and the second end 122 with the back of the housing 11 forms an opened structure, so that the belt body 20 can be disposed between the second end 122 and the back of the housing 11 against the inner wall of the enclosed structure, and then the connection between the timer 10 and the belt body 20 can be achieved by clipping the belt body 20 via the opened structure.

In addition, a plurality of bulges 110 are further disposed on the back of the housing 11, which is disposed corresponding to the connecting component 12, so that the belt body 20 can be clamped between the plurality of bulges 110 and the connecting component 12. Also, since the plurality of bulges 110 can enhance the friction with the surface of the belt body 20, the timer 10 is not easy to slide on the belt body 20 or fall off from the belt body 20. As shown in FIG. 3A, the connecting component 12 may be a clip, but is not limited thereto. In at least one embodiment, an elevated portion 111 may be further disposed between the bulges 110 and the housing 11, which is used to make the belt body 20 attach to the inner wall of the connecting component 12 and/or the bulges 110 more tightly, so as to improve the friction of the contacted surface and to prevent the timer 10 from sliding on the belt body 20 or falling off from the belt body 20. The elevated portion 111 may be, for example, a wedge having a bottom surface, a side surface, and an inclined surface. The bottom surface is connected to the housing 11, and the bulges 110 are disposed on the inclined surface.

FIG. 3B illustrates a tourniquet device in another embodiment of the present disclosure, which is varied based on the embodiments shown in FIGS. 1, 2, and 3A. The difference thereof is the dimension distribution of the plurality of bulges 110, so that the same parts as the aforementioned contents are not repeated herein. In at least one embodiment, the dimension of the bulges 110 approaching the first end 121 is greater than the dimension of the bulges 110 approaching the second end 122, such that the belt body 20 can be tightly attached to the inner wall of the connecting component 12 or the bulges 110. Also, the contact area between the belt body 20 and the inner wall of the connecting component 12 can be increased, so as to improve the friction of the contact surface and to prevent the timer 10 from sliding on the belt body 20 or falling off from the belt body 20.

For example, as shown in FIG. 3B, the dimension of the bulges 110 are gradually reduced in the direction from the first end 121 to the second end 122. Therefore, in comparison to FIG. 3A (the dimension of each bulge 110 is the same), the belt body 20 of FIG. 3B can be attached to the inner wall of the connecting component 12 and the bulges 110 more tightly.

In at least one embodiment of the present disclosure, inside of the housing 11 of the timer 10 has a timing unit and a warning unit, wherein the warning unit is at least one selected from the group consisting of a vibrator, a loudspeaker, and an illuminator. For example, the timer 10 can concurrently have a vibrator, loudspeaker, and illuminator. In an embodiment, the user can turn on the timing unit when using the tourniquet device. The timing of the timing unit may range from 3 minutes to 20 minutes depending on the usage; for example, for blood drawing or injection, the timing may be set at 3 minutes; for stanching bleeding after dialysis, the timing may be set at 20 minutes, of which the present disclosure is not limited thereto. If the timing expires, the vibrator, the loudspeaker, and the illuminator will produce warning vibration, warning sound, and warning light, respectively. The warning sound may be a voice, a tune, a single tone, or any combination of two of them, and further can be defined by specific frequency, rising time or declining time of amplitude, waveform, volume, pulse width, repetition rate, or harmonic of each sound. For instance, the warning sound may have frequencies in the range such as 20 kHz or less, 20 Hz to 20 kHz, 0.5 kHz to 10 kHz, 4 kHz or less, 0.5 kHz to 4 kHz, or 2 kHz to 3 kHz, e.g., but not be limited to, a basic beep, a single tone, a tune, or a voice having a frequency of 20 kHz, 15 kHz, 10 kHz, 9 kHz, 8 kHz, 7 kHz, 6 kHz, 5 kHz, 4.0 kHz, 3.9 kHz, 3.8 kHz, 3.7 kHz, 3.6 kHz, 3.5 kHz, 3.4 kHz, 3.3 kHz, 3.2 kHz, 3.1 kHz, 3.0 kHz, 2.9 kHz, 2.8 kHz, 2.7 kHz, 2.6 kHz, 2.5 kHz, 2.4 kHz, 2.3 kHz, 2.2 kHz, 2.1 kHz, 2.0 kHz, 1 kHz, 0.5 kHz, or 20 Hz. Furthermore, short, fast, and rapid audio changes and combinations thereof may be used to distinguish background sounds produced by many other instruments within intensive care units for users to quickly identify the warning sound. The warning sound may further have a volume/loudness of 75 dB±10% (i.e., 67.5 dB to 82.5 dB) to meet clinical needs and regulations on noise prevention in the workplace. In other embodiments, the warning sound can be selected to have a volume/loudness in the range of 90 dB or less, 60 dB to 90 dB, or 70 dB to 90 dB, e.g., but not be limited to, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, or 60 dB. The warning sound may also be set according to ISO/IEC 60601-1-8 “General requirement, tests, and guidelines for alarm systems in medical electrical equipment and medical electrical system.” The illuminator comprises red LED and green LED, and as shown in FIGS. 1 and 2, the warning light may emit away from the surface of the housing 11 through light pipes 14. Moreover, depending on the states of uses, the illuminator can emit one-color light or two-color light, and the light may be emitted in a manner of constant light, fast flashing, slow flashing, or a combination thereof. For example, the “green light” indicates that the tourniquet device is working; the “interlaced red light and green light” indicates that the power of the tourniquet device is insufficient; and the “red light” indicates a warning that the tourniquet device should be taken off from the patient. The warning unit may further produce a warning related to device abnormalities, such as abnormal conditions of insufficient pressure or connection failure.

As shown in FIGS. 1 and 2, a button 13 is disposed on the front of the housing 11 of the timer 10, and the user can immediately turn on or turn off the timing unit and/or the warning unit inside the timer 10 by pressing the button 13. In at least one embodiment of the present disclosure, the button 13 may be a concave button that is recessed inward. In at least one embodiment of the present disclosure, the center of the button 13 is the most concave part, whereby to prevent the user from accidentally touching the button 13 to turn on or turn off the timing unit and/or the warning unit. In at least one embodiment of the present disclosure, the timer 10 only has a single button 13 to be operated by the user, e.g., pressing the button 13 once to start the timing, and pressing the button 13 again to pause the timing or cancel the warning. In addition, the way to press the button 13 such as pressing the button 13 continuously twice or pressing and holding the button 13 may further be set to provide different action modes.

In at least one embodiment of the present disclosure, the outer housing of the timer 10 of the tourniquet device 1 may be elliptical, oval, arc, etc., without edges and corners, so as to be held in convenience. As shown in FIG. 1, the width “w” of the timer 10 is less than or equal to the width “W” of the belt body 20. For instance, the width w of the timer 10 can be less than or equal to 30 mm, e.g., but not be limited to 30 mm, 29 mm, 28 mm, 27 mm, 26 mm, 25 mm, 24 mm, 23 mm, 22 mm, 21 mm, 20 mm, 19 mm, 18 mm, 17 mm, 16 mm, 15 mm, 14 mm, 13 mm, 12 mm, 11 mm, or 10 mm. The length “l” of the timer 10 can be less than or equal to 55 mm, e.g., but not limited to 55 mm, 54 mm, 53 mm, 52 mm, 51 mm, 50 mm, 49 mm, 48 mm, 47 mm, 46 mm, 45 mm, 44 mm, 43 mm, 42 mm, 41 mm, 40 mm, 39 mm, 38 mm, 37 mm, 36 mm, 35 mm, 34 mm, 33 mm, 32 mm, 31 mm, or 30 mm. The thickness “t” of the timer 10 can be less than or equal to 10 mm, e.g., but not limited to 10 mm, 9 5 mm, 9 mm, 8.5 mm, 8 mm, 7.5 mm, 7 mm, 6 5 mm, 6 mm, 5.5 mm, 5 mm, 4.5 mm, or 4 mm, e.g., less than or equal to 7 mm. In at least one embodiment of the present disclosure, the length l of the timer 10 may be less than or equal to 55 mm, the width w of the timer 10 may be less than or equal to 30 mm, and the thickness t of the timer 10 may be less than or equal to 10 mm

In at least one embodiment of the present disclosure, as shown in FIG. 1, the position of the timer 10 on the belt body 20 can be a position at a distance “L” from the terminal of the belt body 20, wherein L can be in the range of 1 cm to 5 cm, e.g., but not limited to 1 cm, 1.5 cm, 2 cm, 2.5 cm, 3 cm, 3.5 cm, 4 cm, 4.5 cm, or 5 cm, and L may also be in the range of 2 cm to 3.5 cm. In at least one embodiment of the present disclosure, L may be 2.54 cm±0.5 cm (i.e., 2.04 cm to 3.04 cm). Hence, when the user fastens the belt body 20, his or her finger can easily press the button 13 on the timer 10 to turn on the timing unit smoothly without changing the original way of operation to the tourniquet. For example, the two terminals of the belt body 20 have a joint portion and a jointed portion, respectively, such as hooks surface and loops surface of a hook and loop fastener. When the user wraps the belt body 20 on a patient and brings the joint portion and the jointed portion to contact with each other to fasten, or the user loosens the belt body 20, the user can concurrently press the button 13 on the timer 10 located 2.54 cm±0.5 cm away from the terminal of the belt body 20 to rapidly turn on the timing unit or dismiss the setup unit.

In at least one embodiment of the present disclosure, the housing 11 of the timer 10 may be made of waterproof material or provided with a waterproof film depending on actual needs, so as to facilitate cleaning, disinfection, and repeated use, and further to prevent abnormal substances invading inside the timer 10.

In at least one embodiment of the present disclosure, a pressing block may be disposed on the belt body 20 to facilitate stanching bleeding with pressure or congestion. In addition, the timer 10 can further comprise button batteries to provide an electricity source. Also, the timer 10 may further comprise at least one selected from the group consisting of a wireless charging unit, a wireless communication unit, and a pressure sensing unit, so as to accomplish various extended functions such as wireless charging, wireless communication, and pressure sensing. For example, the timing data of the timer 10 may be transmitted to a remote device via a wireless communication unit, such that a person who carries the remote device or is at the position of the remote device can obtain the timing data or confirm whether the belt body 20 provides appropriate pressure to the pressurized part by the pressure sensing unit disposed inside the timer 10.

In at least one embodiment of the present disclosure, the timer 10 with a waterproof housing 11 is clamped and fixed on the belt body 20 by the connecting component 12 thereon, so that the timer 10 and the belt body 20 can be separated easily for cleaning and disinfection by the user after the use. In addition, a plurality of bulges are provided on the back of the housing 11 of the timer 10, such that the plurality of bulges can strengthen the surface friction and tightness between the belt body 20 and the connecting component 12 and/or the housing 11 when the timer 10 is clamped and fixed with the belt body 20 by the connecting component 12 thereon, and such that the timer 10 is not easy to slide on the belt body 20 or fall off from the belt body 20. Therefore, the tourniquet device 1 of the present disclosure is not only lightweight, low cost, effective in warning, but also easier for medical professionals or patients and their caregivers to operate.

What is claimed is:

1. A tourniquet device, comprising:
a belt body; and
a timer, including:
a housing having a plurality of bulges;
a connecting component arranged on outside of the housing to connect the belt body to the timer; and
a timing unit and a warning unit arranged inside the housing,
wherein the belt body is clamped between the bulges and the connecting component.

2. The tourniquet device according to claim 1, wherein the housing is provided with a recessed button to turn on or turn off at least one of the timing unit and the warning unit.

3. The tourniquet device according to claim 1, wherein the timer has a width less than or equal to a width of the belt body.

4. The tourniquet device according to claim 1, wherein the connecting component has a first end and a second end opposite to the first end, and wherein the first end is fixed with the housing and a dimension of the bulges approaching the first end is greater than a dimension of the bulges approaching the second end.

5. The tourniquet device according to claim 1, wherein the timer is positioned at 1 cm to 5 cm from a terminal of the belt body.

6. The tourniquet device according to claim 1, wherein the housing includes water-proof material.

7. The tourniquet device according to claim 1, wherein the warning unit is at least one selected from the group consisting of a vibrator, a loudspeaker, and an illuminator.

8. The tourniquet device according to claim 7, wherein the loudspeaker produces a sound having a frequency below 20 kHz and/or a loudness below 90 dB, and the illuminator includes a red light-emitting diode and a green light-emitting diode.

9. The tourniquet device according to claim 1, wherein the belt body is provided with a pressing block.

10. The tourniquet device according to claim 1, wherein the timer further comprises at least one selected from the group consisting of a wireless charging unit, a wireless communication unit, and a pressure sensing unit.
